(19) United States
(12) Patent Application Publication
(10) Pub. No.: US 2022/0296781 A1
(43) Pub. Date: Sep. 22, 2022

(54) Injectable Temperature-sensitive Composite Hydrogel Containing Adipose-derived Mesenchymal Stem Cells and Preparation Method and Application Thereof

(71) Applicant: Hangzhou Singclean Medical Products Co., Ltd, Hangzhou (CN)

(72) Inventors: Qiang Zhang, Hangzhou (CN); Mengjun Hu, Hangzhou (CN); Feifei Wu, Hangzhou (CN); Wei Huang, Hangzhou (CN); Weiqing Sun, Hangzhou (CN)

(21) Appl. No.: 17/335,039

(22) Filed: May 31, 2021

(30) Foreign Application Priority Data
Mar. 19, 2021 (CN) 202110295199.5

(51) Int. Cl. A61L 27/48; A61L 27/52; A61L 27/38

(52) U.S. Cl. CPC A61L 27/48; A61L 27/52; A61L 27/3834; A61L 27/3852; A61L 2400/06; A61L 2430/06

(57) ABSTRACT

The present application provides an injectable temperature-sensitive composite hydrogel containing adipose-derived mesenchymal stem cells, and a preparation method and application thereof. The present application includes: preparing hydroxypropyl chitin from chitin through modification, preparing a composite with collagen and sodium hyaluronic, constructing the injectable temperature-sensitive composite hydrogel, loading adipose-derived mesenchymal stem cells of New Zealand rabbit and Genipin, and finally forming in-situ the injectable temperature-sensitive composite hydrogel containing adipose-derived mesenchymal stem cells at the physiological temperature. The hydrogel prepared in the present application is of a three-dimensional porous structure, which is conducive to transferring of nutrients and metabolic waste, so as to provide an excellent microenvironment for the growth of cells, helping maintain survival rate and biological activity of the adipose-derived mesenchymal stem cells, and promoting differentiation of the adipose-derived mesenchymal stem cells into cartilage tissue, while having high mechanical strength, and thus can be widely used in cartilage tissue engineering.

### CROSS REFERENCE TO RELATED APPLICATION

The present application claims the benefit of Chinese Patent Application No. 202110295199.5 filed on Mar. 19, 2021, the contents of which are incorporated herein by reference in their entirety.

### TECHNICAL FIELD OF THE INVENTION

The present application relates to the technical field of biomedical materials and tissue engineering, in particular to an injectable temperature-sensitive composite hydrogel containing adipose-derived mesenchymal stem cells and preparation method and application thereof.

### BACKGROUND OF THE INVENTION

Articular cartilage injury is a common orthopedic disease with poor prognosis and high recurrence rate, which brings inconvenience to daily lives and mental pain to tens of millions of patients all over the world. Fracture and improper treatment after fracture, severe wound, infection and bone tumor are the main causes of articular cartilage injury. For early or mild cartilage injury, treatment with drugs can allow the defect to repair itself. But it needs to find more effective treatment when the damaged cartilage is severely defective. Although autologous or allogeneic cartilage transplantation is an effective method to repair cartilage defects, it also has obvious disadvantages, such as secondary injury, limited tissue source, mismatch of donor and recipient tissue in size and shape, damage or dysfunction of the donor site, etc. Therefore, it is urgent to find a simple and effective way to treat cartilage defects.

Tissue engineering techniques based on scaffold materials and adipose-derived mesenchymal stem cells provide a new approach for the treatment of cartilage defects. In recent years, the application of injectable temperature-sensitive hydrogel in scaffold materials has attracted much attention. Its advantage is that hydrogel loaded with adipose-derived mesenchymal stem cells is injected into the affected area through minimally invasive intervention, and then performs self-adjustment according to the size and shape of the defect to form a filler with a specific shape to match the defective cavity. In addition, the injectable temperature-sensitive hydrogel can be formed in situ at the physiological temperature to avoid cell loss. Chitin is a kind of natural polymer material with excellent properties, and the temperature-sensitive hydrogel prepared by chitin have the potential to be used as tissue engineering scaffold materials, but their applications are limited by the low mechanical strength. Therefore, the Chinese patent (CN 108310460 A) uses Genipin to cross-link temperature-sensitive chitin hydrogel. Although the strength of the hydrogel is significantly improved after cross-linking, the introduction of the cross-linking agent weakens the cellular compatibility of the hydrogel, thus limiting its application in cartilage tissue engineering. The simplest and most effective way to solve this problem is to introduce bioactive substances that can promote cell proliferation, such as collagen and hyaluronic acid. Multiple studies have shown that collagen and hyaluronic acid can promote cell proliferation and tissue regeneration, and loading adipose-derived mesenchymal stem cells can further improve the efficiency of tissue regeneration (Zhang et al., 2019, 19:155.; Zhou et al., 2018, 71:496-509.; Yang et al., 2017, 57:1-25.). Therefore, the key problems to be solved are to construct an injectable temperature-sensitive hydrogel containing adipose-derived mesenchymal stem cells, to coordinate the mechanical strength of the hydrogel, to improve its biocompatibility with cells and to ensure the regeneration function of cartilage tissue.

### SUMMARY OF THE INVENTION

In view of the foregoing, the present application provides an injectable temperature-sensitive composite hydrogel containing adipose-derived mesenchymal stem cells and preparation method and application thereof, so as to solve the problems of low survival rate and weak activity of stem cells in the prior art.

In order to achieve the purpose of the first aspect as stated above, the present application adopts the following technical solution: an injectable temperature-sensitive composite hydrogel containing adipose-derived mesenchymal stem cells, wherein the composite hydrogel material comprises adipose-derived mesenchymal stem cells, hydroxypropyl chitin, collagen and sodium hyaluronate.

The ratio of collagen to sodium hyaluronate is preferably (0.1%-3%):(0.1%-0.6%).

The ratio of hydroxypropyl chitin to collagen to sodium hyaluronate is preferably (1.2%-4%):(0.1%-3%):(0.1%-0.6%), the content of the adipose-derived mesenchymal stem cells is 1×10⁶-1×10⁸ cells/mL.

The injectable temperature-sensitive composite hydrogel of the present application not only comprises the above-mentioned main components of adipose-derived mesenchymal stem cells, hydroxypropyl chitin, collagen and sodium hyaluronate, but can also comprise polypeptides, oligopeptides, amino acids or bioactive substances with cell growth promoting effects.

In order to achieve the purpose of the first aspect as stated above, the present application adopts the following technical solution: a preparation method of an injectable temperature-sensitive composite hydrogel containing adipose-derived mesenchymal stem cells, and the preparation method comprises the following steps:

S1. dissolving chitin in a mixed alkali solution of sodium hydroxide and urea, adding epoxypropane to prepare hydroxypropyl chitin, and performing dialysis, drying and dissolution to obtain a hydroxypropyl chitin solution.

S2. mixing the hydroxypropyl chitin solution with a collagen solution and a sodium hyaluronate solution to prepare a composite solution.

S3. re-suspending adipose-derived mesenchymal stem cells with the composite solution prepared in S2, and obtaining the injectable temperature-sensitive composite solution containing adipose-derived mesenchymal stem cells under stirring.

S4. adding Genipin to the injectable temperature-sensitive composite solution prepared in S3 to obtain a mixture, and then placing the mixture at appropriate corresponding gelation temperature, to obtain the injectable temperature-sensitive composite hydrogel containing adipose-derived mesenchymal stem cells.

By making a composite of hydroxypropyl chitin with collagen and sodium hyaluronate, the preparation method of the injectable temperature-sensitive hydrogel of the present application is conducive to improving the activity and survival rate of stem cells in the hydrogel. The injectable temperature-sensitive composite hydrogel containing adipose-derived mesenchymal stem cells is constructed by loading adipose-derived mesenchymal stem cells. The hydrogel solidification was realized by in-situ molding at physiological temperature to provide a stable growth environment for cells, thus improving the tissue regeneration ability of hydrogel. The specific preparation methods mentioned above can not only improve the survival rate and activity of stem cells, so as to promote regeneration of cartilage tissue, but also improve the mechanical strength of the hydrogel, so that the hydrogel can better support the rapid proliferation of cells and withstand external forces.

Preferably, the molecular weight of chitin in S1 ranges from 5,000-1,000,000 and the molecular weight of collagen and sodium hyaluronate in S2 ranges from 50,000-300,000 and 80,000-2,300,000, respectively. The direct technical effect brought by this optimization solution is that the composite solution is not too viscous to affect the preparation process of hydrogel, and not too thin to form a gel, which is more conducive to the control of preparation technology and the steady progress of continuous production.

Preferably, the mass ratio of hydroxylpropyl chitin, collagen and sodium hyaluronate in the composite solution prepared in S2 is (15-20):(1-3):(1-3).

Preferably, in S3, the injectable temperature-sensitive composite solution contains adipose-derived mesenchymal stem cells with a concentration of 1×10⁶-1×10⁸/mL.

In the preparation method, polypeptides, oligopeptides, amino acids or bioactive substances with cell growth promotion effects can be mixed, along with the hydroxypropyl chitin solution, with the collagen solution and the sodium hyaluronate solution in S2 to prepare a composite solution.

In the preparation method, epoxypropane is added to undergo reaction under the following conditions for preparation of hydroxypropyl chitin: first of all, in an ice bath, performing mechanical stirring of the reactants for reaction for 0.5-2 h, then heating up to 4-10° C. for reaction for 12-36 h, heating up to 15-34° C. for reaction for 1-12 h, finally cooling to 0-10° C. and standing for 1-12 h. The method can prevent adverse effect on the reaction due to constant volatilization of epoxypropane in the process of stirring, and can prevent the generated hydroxypropyl chitin from curing, thus allowing the reaction to proceed smoothly, as a result, the whole reaction is more complete and the gel has better temperature sensitivity. In the present application, the gelation temperature is 37° C.

In the preparation method, the drying methods of hydroxypropyl chitin can be natural drying, blast drying, freeze drying, vacuum drying and critical point drying.

In another embodiment of the present application, the concentration of Genipin in the injectable temperature-sensitive composite hydrogel containing adipose-derived mesenchymal stem cells obtained in step S4 is 0.01-0.05 wt %. It is conducive to preparing hydrogel with different mechanical strengths by further defining Genipin.

The mechanical strength of the injectable temperature-sensitive composite hydrogel containing adipose-derived mesenchymal stem cells obtained by the method is 2343.6-2490.8 Pa, and the gelation time at 37° C. is 2.7-4.2 min, and can be used as a material for cell scaffolds and cartilage repair scaffolds.

The preparation method application and the injectable temperature-sensitive composite hydrogel of the present application have the following advantages:

(1) The present application is conducive to improving the activity and survival rate of stem cells in the hydrogel by preparing an injectable temperature-sensitive hydrogel with a composite of hydroxypropyl chitin with collagen and sodium hyaluronate. An injectable temperature-sensitive composite hydrogel containing adipose-derived mesenchymal stem cells is constructed by loading adipose-derived mesenchymal stem cells. The hydrogel solidification is realized by in-situ molding under physiological temperature to provide a stable growth environment for cells, thus improving the tissue regeneration ability of hydrogel.

(2) The specific preparation method of the application can not only improve the survival rate and activity of stem cells, so as to facilitate regeneration of cartilage tissue, but also improve the mechanical strength of hydrogel, so that the hydrogel can better support the rapid proliferation of cells and withstand external forces.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows a sample picture and an injection diagram of injectable temperature-sensitive composite hydrogel obtained in Examples 3-8.

FIG. 2 shows the histogram of the statistical results of the average pushing force of the injectable temperature-sensitive composite hydrogel samples with different ratios obtained in Examples 3-8.

FIG. 3 shows photos of states of the injectable temperature-sensitive composite hydrogel samples prepared via gelation at different temperatures in Examples 3-8, wherein a, b and c show the conditions of the sample at 4° C., 25° C. and 37° C., respectively.

FIG. 4 shows the histogram of the statistical results of gelation time at different temperatures with the mass ratios of Examples 3-8, wherein B and C show the gelation time of each sample at 25° C. and 37° C., respectively.

FIG. 5 shows the scanning electron microscopy of the composite hydrogel with different ratios obtained in Examples 3-8, wherein a, b, c, d, e and f represent composite hydrogels containing hydroxypropyl chitin, collagen, and sodium hyaluronate with a mass ratio of 20:1:2, 20:2:1, 20:1:1, 15:2:2, 15:3:1 and 15:1:3, respectively.

FIG. 6 shows the statistical results of compression strength before (A) and after (B) crosslinking with Genipin under the conditions of different ratios in Examples 3-8.

FIGS. 7 and 8 respectively show the proliferation and fluorescence staining of adipose-derived mesenchymal stem cells in the composite hydrogel with different ratios obtained in Examples 2-8. FIG. 7 shows the absorbance values of the culture system after 1, 2 and 3 days of culture. FIG. 8 shows the fluorescence staining of cells after 3 days of culture, a represents pure hydroxypropyl chitin hydrogel, and b, c, d, e, f and g respectively represent composite hydrogel containing hydroxypropyl chitin, collagen, and sodium hyaluronate with a mass ratio of 20:1:2, 20:2:1, 20:1:1, 15:2:2, 15:3:1 and 15:1:3.

FIG. 9 shows the immunofluorescence staining of the composite hydrogel with different ratios obtained in Examples 1-8 cultured in an incubator at 37° C. in 5% carbon dioxide for 21 consecutive days. A and B are respectively immunofluorescence staining diagrams of type II collagen and type I collagen, a is hydroxypropyl chitin hydrogel without adipose-derived mesenchymal stem cells, b is hydroxypropyl chitin hydrogel containing adipose-derived mesenchymal stem cells, c, d, e, f, g and h respectively represent composite hydrogel containing hydroxypropyl chitin, collagen and sodium hyaluronate with a mass ratio of 20:1:2, 20:2:1, 20:1:1, 15:2:2, 15:3:1 and 15:1:3.

FIG. 10 shows the immunohistochemical staining results of the composite hydrogel with different ratios obtained in Examples 1-8 cultured in an incubator at 37° C. in 5% carbon dioxide for 21 days, a is hydroxypropyl chitin hydrogel without adipose-derived mesenchymal stem cells, b is hydroxypropyl chitin hydrogel containing adipose-derived mesenchymal stem cells, c, d, e, f, g and h respectively represent composite hydrogel containing hydroxypropyl chitin, collagen and sodium hyaluronate with a mass ratio of 20:1:2, 20:2:1, 20:1:1, 15:2:2, 15:3:1 and 15:1:3.

### DETAILED DESCRIPTION OF THE INVENTION

The embodiments of the present application are illustrated with the following specific examples. The person skilled can easily understand other advantages and effects of the present application from the contents indicated in this specification. Obviously, the examples described herein are parts rather than all examples of the present application. On the basis of the examples in the present application, all other examples obtained by those skilled in the art without making creative efforts fall into the protection scope of the present application.

The raw materials used in the following examples are as follows:

Chitin powder: having a molecular weight of 5,000-1,000,000, from Shanghai Aladdin Biochemical Technology Co., Ltd.

Collagen: having a molecular weight of 50,000-300,000, from Hangzhou Singclean Medical Products Co., Ltd.

Sodium hyaluronate: having a molecular weight of 80,000-2,300,000, from BLOOMAGE BIOTECHNOLOGY CORPORATION LIMITED.

Epoxypropane, sodium hydroxide and urea: from Sinopharm Chemical Reagents Co., Ltd.

Adipose-derived mesenchymal stem cells: derived from the groin of New Zealand rabbit, and provided by Cyagen Biosciences Inc.

### EXAMPLE 1

The present example is a preparation method of an injectable temperature-sensitive hydroxypropyl chitin hydrogel, which comprises the following steps:

S1. chitin powder was dissolved in a mixed alkali solution containing 11 wt % sodium hydroxide and 4 wt % urea, epoxypropane was added therein, then in an ice bath, the mixture was mechanically stirred for reaction for 2 h, then heated to 5° C. for reaction for 24 h, heated to 15° C. for reaction for 6 h, and finally cooled to 4° C. and was allowed to stand for 2 h, to prepare hydroxypropyl chitin. And hydroxypropyl chitin underwent dialysis, freeze drying and dissolution to obtain a 2.5 wt % hydroxypropyl chitin solution.

S2. Genipin was added to the hydroxypropyl chitin solution of S1, and the mixture was placed at 37° C. to obtain an injectable temperature-sensitive hydroxypropyl chitin hydrogel, which contains Genipin with a concentration of 0.02 wt %.

### EXAMPLE 2

The present example is a preparation method of an injectable temperature-sensitive hydroxypropyl chitin hydrogel containing adipose-derived mesenchymal stem cells. The preparation method comprises the following steps:

S1. chitin powder was dissolved in a mixed alkali solution containing 11 wt % sodium hydroxide and 4 wt % urea, epoxypropane was added therein, then in an ice bath, the mixture was mechanically stirred for reaction for 2 h, then heated to 5° C. for reaction for 24 h, heated to 15° C. for reaction for 6 h, and finally cooled to 4° C. and was allowed to stand for 2 h to prepare hydroxypropyl chitin. And hydroxypropyl chitin underwent dialysis, freeze drying and dissolution to obtain a 2.5 wt % hydroxypropyl chitin solution.

S2. adipose-derived mesenchymal stem cells were re-suspended with the hydroxypropyl chitin solution prepared in S1, and the injectable temperature-sensitive hydroxypropyl chitin solution containing adipose-derived mesenchymal stem cells was obtained under stirring, in which the cell concentration of the adipose-derived mesenchymal stem cells was 1×10⁶/mL.

S3. Genipin was added to the injectable temperature-sensitive hydroxypropyl chitin solution of S2, and the mixture was placed at 37° C. to obtain an injectable temperature-sensitive hydroxypropyl chitin hydrogel containing adipose-derived mesenchymal stem cells, which contains Genipin with a concentration of 0.02 wt %.

### EXAMPLE 3

The present example is a preparation method of an injectable temperature-sensitive composite hydrogel containing adipose-derived mesenchymal stem cells, which comprises the following steps:

S1. chitin powder was dissolved in a mixed alkali solution containing 11 wt % sodium hydroxide and 4 wt % urea, epoxypropane was added therein, then in an ice bath, the mixture was mechanically stirred for reaction for 2 h, then heated to 5° C. for reaction for 24 h, heated to 15° C. for reaction for 6 h, and finally cooled to 4° C. and allowed to stand for 2 h to prepare hydroxypropyl chitin. And hydroxypropyl chitin underwent dialysis, freeze drying and dissolution to obtain a 2.5 wt % hydroxypropyl chitin solution.

S2. the hydroxypropyl chitin solution was mixed with a collagen solution and a sodium hyaluronate solution to prepare a composite solution, in which the concentration of the collagen solution was 1 wt %, and the concentration of the sodium hyaluronate solution was 1 wt %. The mass ratio of hydroxypropyl chitin, collagen and sodium hyaluronate in the composite solution was 20:1:2.

S3. adipose-derived mesenchymal stem cells were re-suspended with the composite solution prepared in S2, and an injectable temperature-sensitive composite solution containing adipose-derived mesenchymal stem cells was obtained under stirring, in which the cell concentration of the adipose-derived mesenchymal stem cells was 1×10⁶/mL.

S4. Genipin was added to the injectable temperature-sensitive composite solution prepared in S3, and then the mixture was placed at 37° C. to obtain an injectable temperature-sensitive composite hydrogel containing adipose-derived mesenchymal stem cells, which contains Genipin with a concentration of 0.02 wt %.

### EXAMPLE 4

The steps were the same as in Example 3, except that “in S2, the mass ratio of hydroxypropyl chitin, collagen, and sodium hyaluronate in the composite solution was 20:2:1”.

### EXAMPLE 5

The steps were the same as in Example 3, except that “in S2, the mass ratio of hydroxypropyl chitin, collagen, and sodium hyaluronate in the composite solution was 20:1:1”.

### EXAMPLE 6

The steps were the same as in Example 3, except that “in S2, the mass ratio of hydroxypropyl chitin, collagen, and sodium hyaluronate in the composite solution was 15:2:2”.

### EXAMPLE 7

The steps were the same as in Example 3, except that “in S2, the mass ratio of hydroxypropyl chitin, collagen, and sodium hyaluronate in the composite solution was 15:3:1”.

### EXAMPLE 8

The steps were the same as in Example 3, except that “in S2, the mass ratio of hydroxypropyl chitin, collagen, and sodium hyaluronate in the composite solution was 15:1:3”.

### TEST EXAMPLE 1

Injectable temperature-sensitive composite hydrogel containing adipose-derived mesenchymal stem cells was prepared according to the Example 3-8 respectively. The prepared hydrogel samples were put into a 1 ml syringe containing a 27 G needle, respectively. The push rod of the syringe was pushed to write on blank paper, while the syringe was clamped on tension test machine (HG1697A, Kunshan Hengguang Instrument Co., LTD., Jiangsu, China), the squeezing speed was adjusted to 10 mm/min, and the average squeezing force in the process of squeezing was recorded.

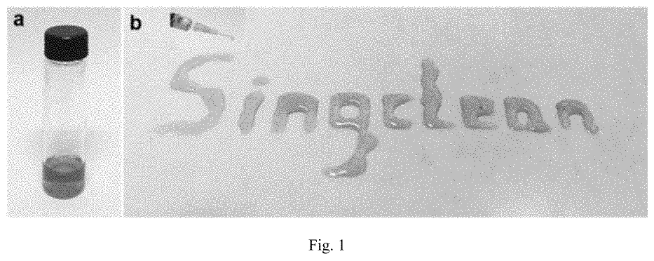

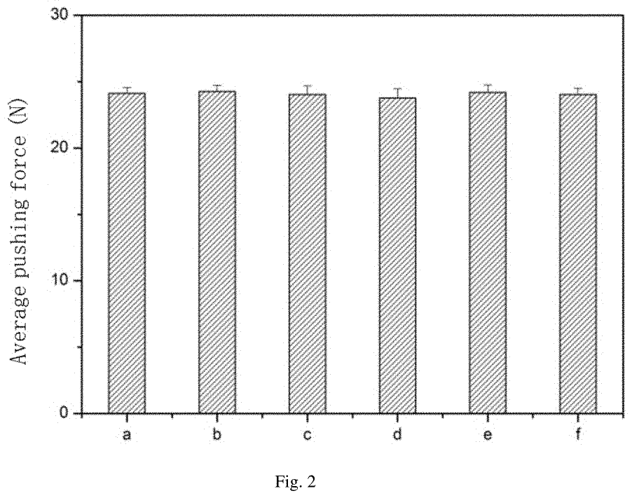

FIG. 1 shows an injection diagram of composite hydrogel obtained in Examples 3-8 with different ratios, and FIG. 2 shows the statistical results of the average squeezing force of the composite hydrogel. As can be seen from the diagram, the prepared hydrogel can write freely via a syringe, with a small average squeezing force throughout the injection process, indicating that the hydrogel has good injection performance.

### TEST EXAMPLE 2

Injectable temperature-sensitive composite hydrogel containing adipose-derived mesenchymal stem cells was prepared according to the above Examples 3-8 respectively, 2 mL of the samples prepared in examples 3-8 were put into a 10 mL glass bottle respectively, and after the bottle cap was screwed tightly, the bottle was placed at 4° C. to stand for 10 min, 25° C. to stand for 10 min, and 37° C. to stand for 10 min. The state of the samples at various temperatures was recorded. The samples were placed in the environments of 4° C., 25° C. and 37° C. successively, and taken out to observe the state every 30 s. If the sample did not peel off when reversed for 30 s, it was determined that a gel had been formed, and the time for gel formation of the samples was recorded.

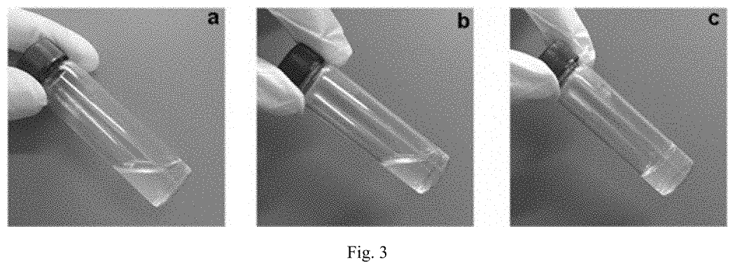

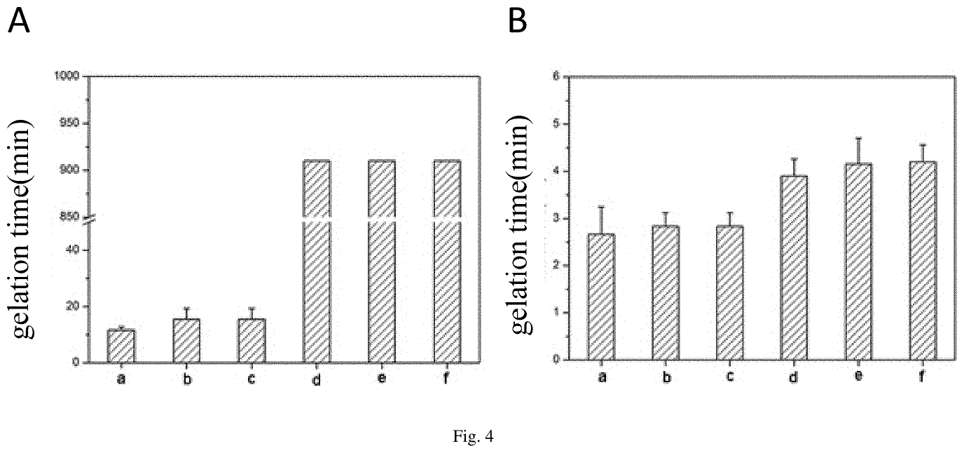

FIG. 3 shows the states of composite hydrogel prepared in Examples 3-8 at different temperatures and with different ratios, and FIG. 4 shows the statistical results of gelation time of each composite hydrogel. As can be seen from FIG. 3, the prepared hydrogel is liquid at 4° C. and 25° C., and solid at 37° C. According to the statistical results of FIG. 4, at 25° C., when the mass ratio of hydroxypropyl chitin, collagen and sodium hyaluronic is 20:1:2, 20:2:1 and 20:1:1, the hydrogel can form a gel within 20 min; and when the mass ratio is 15:2:2, 15:3:1 and 15:1:3, the hydrogel forms a gel after 900 min; and at 37° C., hydrogel of all ratios can form a gel within 5 min, demonstrating that the prepared hydrogel can quickly form a gel at physiological temperature, and the gelation time of the hydrogel can be adjusted by changing the proportion of hydroxypropyl chitin.

### TEST EXAMPLE 3

Injectable temperature-sensitive composite hydrogel containing adipose-derived mesenchymal stem cells are prepared according to the Examples 3-8 respectively, 100 μL of the samples prepared in Examples 3-8 were put into a 24-well culture plate, frozen at −20° C. for 2 h, freeze-dried in a freeze-dryer for 24 h, and was sprayed with metal before the surface morphology of the samples was observed by a scanning electron microscope.

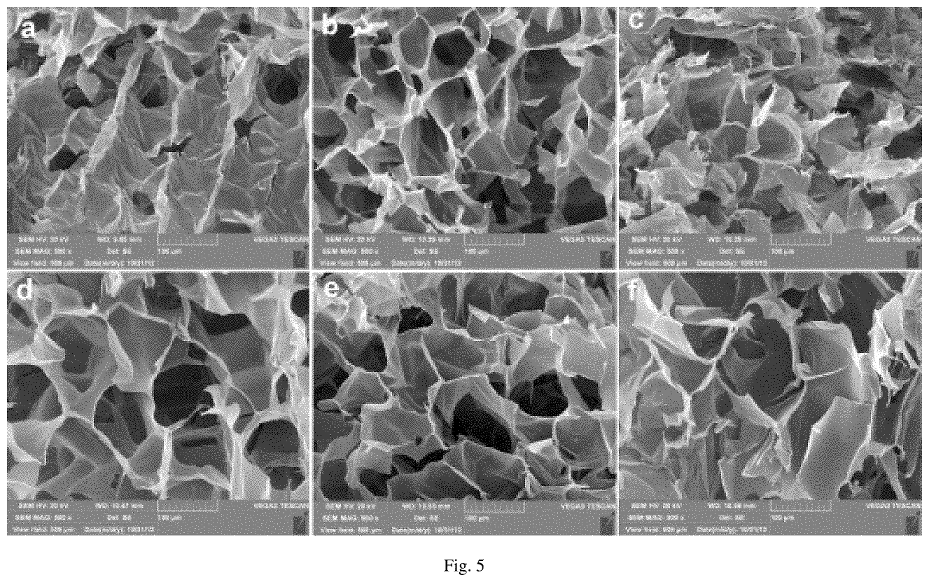

FIG. 5 shows scanning electron microscopy of composite hydrogel with different ratios obtained in Examples 3-8. It can be seen from the figure that each prepared hydrogel was of a three-dimensional porous structure, which was conducive to the exchange of nutrients and metabolic wastes.

### TEST EXAMPLE 4

Injectable temperature-sensitive composite hydrogel containing adipose-derived mesenchymal stem cells was prepared according to the Examples 3-8 respectively, the compression stress rate was adjusted to 0.05 N/min using a dynamic mechanical analyzer (DMA, TA Instrument Q800 series, USA). The samples prepared in Examples 2-6 were analyzed, and the compression strength of each sample was measured and recorded at 37° C.

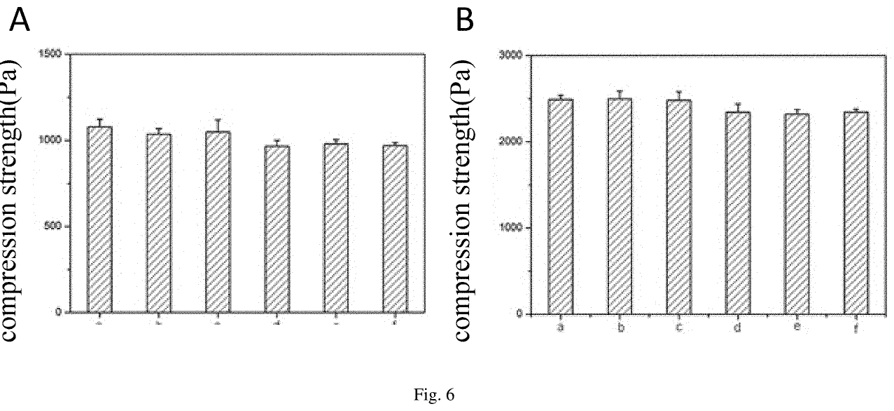

FIG. 6 shows the statistical results of compression strength of the composite hydrogel obtained in Examples 3-8 with different ratios. It can be seen from the figure that the compressive strength of the hydrogel without cross-linking by Genipin was 970-1078 Pa, while the compressive strength of the hydrogel with cross-linking by Genipin was more than 2300 Pa, indicating that the compressive strength of the hydrogel can be significantly improved by cross-linking with Genipin.

### TEST EXAMPLE 5

Injectable temperature-sensitive composite hydrogel containing adipose-derived mesenchymal stem cells was prepared according to the above Examples 2-8 respectively, 200 μL of the samples prepared in Examples 2-8 of were put into a 24-well culture plate, incubated at 37° C. for 30 min, and after refilling of 800 μL cell culture medium, the samples continued to be cultivated at 37° C. in 5% carbon dioxide, 10 μL of PrestoBlue™ cell activity reagent (HH-A13262, Life Technologies, USA) was added on the first, second and third day, respectively, after incubation at 37° C. for a further 30 min, 100 μL of the culture supernatant was sucked out. The absorbance value of each supernatant at 570 nm was recorded with a microplate reader, the samples on the third day were stained, and the cell state was observed with an inverted fluorescence microscope.

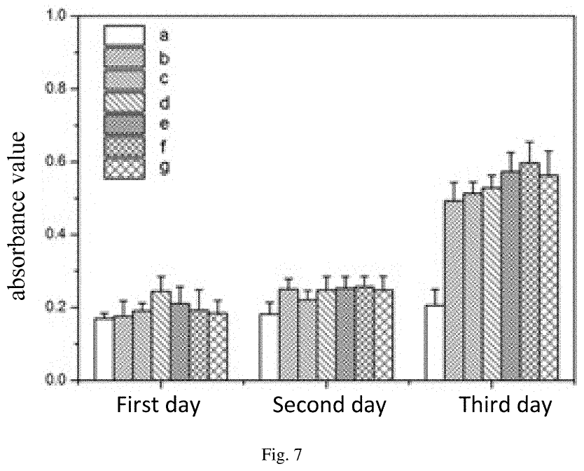

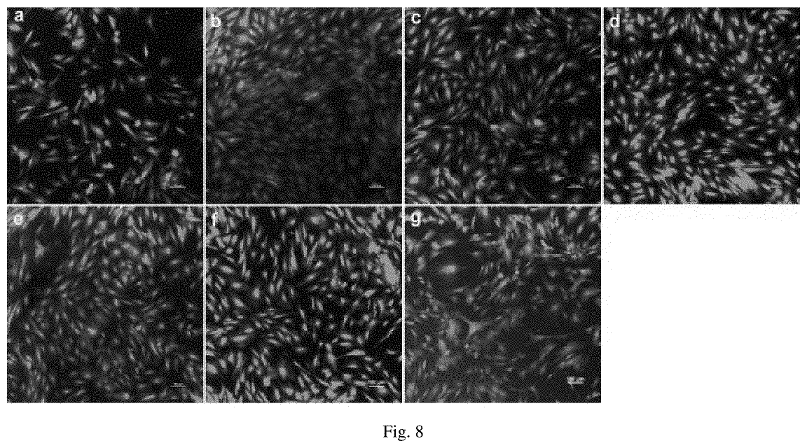

FIGS. 7 and 8 respectively show the proliferation state and fluorescence staining of the adipose-derived mesenchymal stem cells in the composite hydrogel with different ratios obtained in Examples 2-8. The figure shows that cells continuously cultured in the pure hydroxypropyl chitin hydrogel for three consecutive days do not show significant proliferation, accompanied by a lot of dead cells, and the cells in the composite hydrogel show remarkable proliferation, with only a few dead cells, indicating that the addition of collagen and sodium hyaluronic can improve the survival rate of the adipose-derived mesenchymal stem cells and enhance the activity thereof.

### TEST EXAMPLE 6

Injectable temperature-sensitive composite hydrogel containing adipose-derived mesenchymal stem cells was prepared according to the above Examples 1-8 respectively, 200 μL of the samples prepared in Examples 1-8 of were put into a 6-well culture plate, incubated at 37° C. for 30 min, and after refilling of 1.8 mL of differentiation medium (Cyagen Biosciences Inc., Jiangsu, China), continued to be cultivated at 37° C. in 5% carbon dioxide for 21 consecutive days, with the culture medium being replaced every 3 days. The samples were taken out, fixed with 2.4% glutaraldehyde for 1 h, and then subjected to immunofluorescence staining.

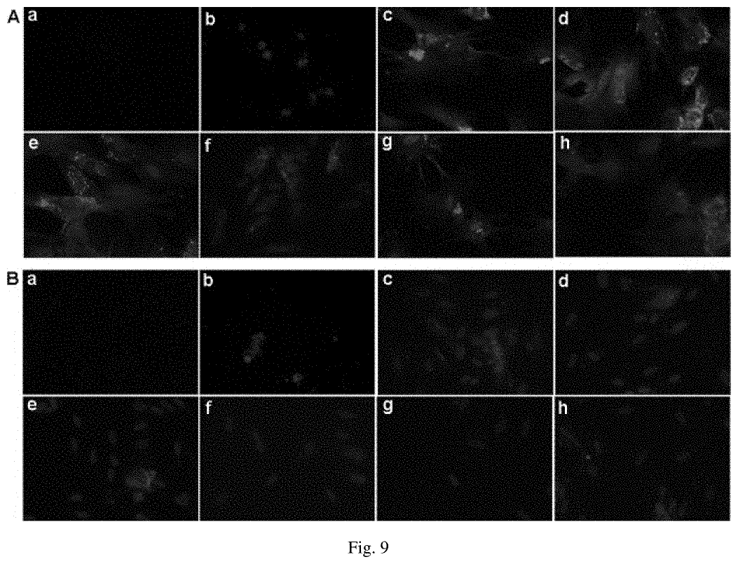

FIG. 9 shows the immunofluorescence staining of the composite hydrogel obtained in Examples 1-8 with different ratios after culturing in an incubator at 37° C. in 5% carbon dioxide for 21 consecutive days. The figure shows that hydrogel without adipose-mesenchymal stem cells does not express type I and type II collagen, hydroxypropyl chitin hydrogel containing adipose-derived mesenchymal stem cells expresses type I collagen at a low level, but does not express type II collagen, and the composite hydrogel containing adipose-derived mesenchymal stem cells can express type II collagen at a high level, but rarely expresses type I collagen. The results indicate that the composite hydrogel can promote the differentiation of the adipose-derived mesenchymal stem cells into cartilage cells.

### TEST EXAMPLE 7

Injectable temperature-sensitive composite hydrogel containing adipose-derived mesenchymal stem cells was prepared according to the above Examples 1-8 respectively, 200 μL of the samples prepared in Examples 1-8 were put into a 24-well culture plate, incubated at 37° C. for 30 min, and after refilling of 1.8 mL of differentiation medium, continued to be cultivated at 37° C. in 5% carbon dioxide for 21 consecutive days, with the culture medium being replaced every 3 days. After 21 days, the formed tissue samples were fixed with 2.4% glutaraldehyde for 48 h and subjected to immunofluorescence staining.

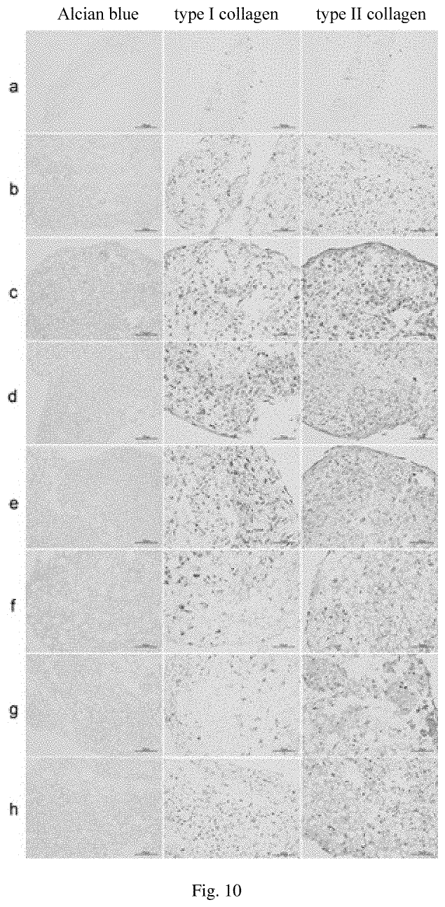

FIG. 10 shows the immunohistochemical staining results of the composite hydrogel obtained in Examples 1-8 with different ratios after culturing in an incubator at 37° C. in 5% carbon dioxide for 21 days. As shown in the figure, the hydrogel without adipose-derived mesenchymal stem cells shows negative for staining of Alcian blue, type I collagen and type II collagen. Hydroxypropyl chitin hydrogel containing adipose-derived mesenchymal stem cells shows positive for Alcian blue staining and type I collagen staining, but negative for type II collagen staining. Composite hydrogel containing adipose-derived mesenchymal stem cells shows positive for Alcian blue staining and type II collagen staining, and negative for type I collagen staining. These results indicate that injectable temperature-sensitive composite hydrogel containing adipose-derived mesenchymal stem cells can promote the formation of cartilage tissue from the cells.

Although the present application has been described in detail with general descriptions and specific examples above, modifications or improvements may be made on the basis of the present application, which is obvious to the person skilled in the field. Therefore, any modification or improvement without departing from the spirit of the present application falls within the protection scope claimed by the present application.

1. An injectable temperature-sensitive composite hydrogel containing adipose-derived mesenchymal stem cells, wherein the composite hydrogel material comprises adipose-derived mesenchymal stem cells, hydroxypropyl chitin, collagen and sodium hyaluronate.

2. The injectable temperature-sensitive composite hydrogel containing adipose-derived mesenchymal stem cells according to claim 1, wherein the ratio of collagen to sodium hyaluronate is preferably (0.1%-3%):(0.1%˜0.6%).

3. The injectable temperature-sensitive composite hydrogel containing adipose-derived mesenchymal stem cells according to claim 1, wherein the ratio of hydroxypropyl chitin to collagen to sodium hyaluronate is preferably (1.2%-4%):(0.1%-3%):(0.1%-0.6%), and the content of the adipose-derived mesenchymal stem cells is 1×10⁶-1×10⁸/mL.

4. A preparation method of the injectable temperature-sensitive composite hydrogel containing adipose-derived mesenchymal stem cells according to claim 1, comprises the following steps:
S1. dissolving chitin in a mixed alkali solution of sodium hydroxide and urea, adding epoxypropane to prepare hydroxypropyl chitin, and performing dialysis, drying and dissolving, to obtain a hydroxypropyl chitin solution,
S2. mixing the hydroxypropyl chitin solution with a collagen solution and a sodium hyaluronate solution to prepare a composite solution,
S3. re-suspending adipose-derived mesenchymal stem cells with the composite solution prepared in S2, and obtaining the injectable temperature-sensitive composite solution containing adipose-derived mesenchymal stem cells under stirring, and
S4. adding Genipin to the injectable temperature-sensitive composite solution prepared in S3 to obtain a mixture, and then placing the mixture at a corresponding gelation temperature, to obtain the injectable temperature-sensitive composite hydrogel containing adipose-derived mesenchymal stem cells.

5. The preparation method according to claim 4, wherein, molecular weights of the used chitin, collagen and sodium hyaluronate are in a range of 5,000-1,000,000, 50,000-300,000 and 80,000-2,300,000, respectively.

6. The preparation method according to claim 4, wherein, the mass ratio of hydroxylpropyl chitin, collagen and sodium hyaluronate in the composite solution prepared in S2 is (15-20):(1-3):(1-3).

7. The preparation method according to claim 4, wherein, the injectable temperature-sensitive composite solution contains adipose-derived mesenchymal stem cells with a concentration of 1×10⁶-1×10⁸/mL in S3.

8. The preparation method according to claim 4, wherein, the concentration of Genipin is 0.01-0.05 wt % in the injectable temperature-sensitive composite hydrogel containing adipose-derived mesenchymal stem cells obtained in S4.

9. An injectable temperature-sensitive composite hydrogel containing adipose-derived mesenchymal stem cells obtained by the method of claim 4, wherein, the hydrogel has a mechanical strength of 2343.6-2490.8 Pa, and a gelation time of 2.7-4.2 min at 37° C.

10. Application of the injectable temperature-sensitive composite hydrogel containing adipose-derived mesenchymal stem cells according to claim 1 as a material for cellular scaffolds and cartilage repair scaffolds.

11. Application of the injectable temperature-sensitive composite hydrogel containing adipose-derived mesenchymal stem cells according to claim 9 as a material for cellular scaffolds and cartilage repair scaffolds.
